(19) United States
(12) Patent Application Publication
(10) Pub. No.: US 2004/0132743 A1
(43) Pub. Date: Jul. 8, 2004

(54) Amorphous form of (-)-[2-[4-[(4-Chlorophenyl)-phenyl methyl]-1- piperazinyl] ethoxy] acetic acid dihydrochloride (levocetirizine dihydrochloride)

(75) Inventors: Manne Satyanarayana Reddy, Hyderabad (IN); Srinivasan Thirumalai Rajan, Hyderabad (IN); Uppala Venkata Bhaskara Rao, Hyderabad (IN); Vaddadi Pattabhi Ramayya, Hyderabad (IN)

Correspondence Address: Ladas & Parry, 26 West 61 Street, New York, NY, 10023, US

(73) Assignee: DR. REDDY'S LABORATORIES LIMITED DR. REDDY'S LABORATORIES, INC.

(21) Appl. No.: 10/601,844

(22) Filed: Jun. 23, 2003

(30) Foreign Application Priority Data
Jun. 21, 2002 (IN) 472/MAS/2002

(51) Int. Cl. C07D 241/04; A61K 31/495

(52) U.S. Cl. 514/255.04; 544/396

(57) ABSTRACT

The present invention relates to the amorphous form of levocetirizine dihydrochloride. The present invention also relates to the process for the preparation of the amorphous form of levocetirizine dihydrochloride. The amorphous form of levocetirizine dihydrochloride is suitable for pharmaceutical purposes in the treatment of allergies, including ailments such as chronic and acute allergic rhinitis, allergic conjunctivitis, pruritus, urticaria and the like.

### CROSS REFERENCE TO RELATED APPLICATIONS

This application claims benefit of a filing date of an Indian Patent Application No. 472/MAS/2002; filed Jun. 21, 2002, the contents of which are expressly incorporated herein by reference.

### FIELD OF THE INVENTION

The present invention relates to the amorphous form of (−)-[2-[4-[(4-Chlorophenyl)-phenyl methyl]-1-piperazinyl]ethoxy] acetic acid dihydrochloride, generically known as levocetirizine dihydrochloride, the process for preparation of the amorphous form of levocetirizine dihydrochloride, and compositions containing the amorphous form of levocetirizine dihydrochloride.

### BACKGROUND OF THE INVENTION

There has been a significant increase in the number of reported allergic conditions over the last three decades. This is evidenced by the increased levels of antibodies developed in response to environmental allergic factors, such as dust mites, pets, and air pollutants. See, e.g., American Journal of Respiratory and Critical Care Medicine, 159:125-29 (1999). Consequently, it is of great importance to develop new drugs that alleviate allergy symptoms.

Cetirizine and its salts, including dihydrochloride, is known and is effective in the treatment of allergies, including but not limited to, chronic and acute allergic rhinitis, allergic conjunctivitis, pruritus, urticaria, and the like. Cetirizine is orally active, long-acting, histamine H₁ receptor antagonist. Antihistamines, such as cetirizine, block the effect of histamines that are released by allergic reactions in the body. This mitigates the ability of histamine to promote allergy symptoms. Cetirizine belongs to the second generation of H₁ histamine receptor antagonists, which are believed to offer significant advantages over first generation compounds. Studies have shown that cetirizine provides safe and effective, symptomatic relief of seasonal allergies. Cetirizine has been shown to provide a positive impact on patients who have experienced activity impairment from allergies; thereby significantly improving health related quality of life. See, e.g., Murray et al., Comprehensive evaluation of Cetirizine in the management of seasonal allergic rhinitis: impact on symptoms, quality of life, productivity, and activity impairment, Allergy Astma Proc., November-December, 23(6): acting duration. Non-sedating character of cetirizine is an important breakthrough in allergy treatment because new generation allergy drugs diminish the commonly experienced, sedative effect and allow patients to enjoy an improved quality of life.

Cetirizine has an asymmetric center in the molecule, which marked with an asterisk in the formula below, and, thus may exist as optical isomers (enantiomers):

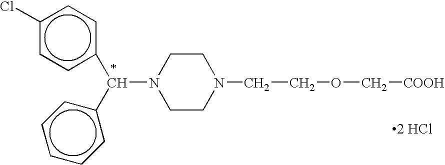

Levocetirizine is the R enantiomer of cetirizine. Levocetirizine, like cetirizine, has a potential anti-inflammatory effect in the treatment of allergic rhinitis with asthma. Levocetirizine is believed to have a two-fold higher affinity for human H₁ receptors than cetirizine. Levocetirizine is believed to be rapidly and extensively absorbed. Levocetirizine also has been shown to be free from side effects on the central nervous system. See, e.g., Journal of Allergy and Clinical Immunology, 111:3: 623-627 (2003).

The preparation of cetirizine generally is known in the art. For example, the process for the preparation of cetirizine and its salts is disclosed U.S. Pat. No. 4,525,358. The disclosed process involves hydrolysis of the methyl ester of cetirizine using ethanolic potassium hydroxide to afford potassium salt of cetirizine. The solution containing the potassium salt is then acidified with hydrochloric acid. U.S. Pat. No. 6,255,487 discloses a process for the preparation of cetirizine dihydrochloride via condensation of (4-chloro phenyl) phenyl methyl chloride and potassium 2-(1-piperazinyl) ethoxyacetate in acetonitrile, followed by acidification in acetone medium with concentrated hydrochloric acid.

EP 58,146 discloses a process for the preparation of cetirizine dihydrochloride, which involves treating cetirizine with methyl (2-chloroethoxy)-acetate which is then subjected to hydrolysis with an inorganic base to produce sodium or potassium salt; it is then converted into free acid species and later into cetirizine dihydrochloride.

Amorphous form of a drug may exhibit different dissolution characteristics and in some cases different bioavailability patterns compared to crystalline forms. See, e.g., Konne T., Chem. Pharm. Bull. 38, 2003 (1990). Also, for some therapeutic indications one bioavailability pattern may be favored over another. For example, amorphous form of cefuroxime axetil exhibits higher bioavailability than its crystalline form. Further, amorphous and crystalline forms of a drug may have different handling properties, dissolution rates, solubility, and stability. For these reasons, among others, access to a choice of amorphous or crystalline form of drug is desirable for different applications.

### SUMMARY OF INVENTION

In accordance with one aspect, the present invention provides an amorphous form of levocetirizine dihydrochloride.

In accordance with another aspect, the invention provides a pharmaceutical composition that includes a prophylactically or therapeutically effective amount of the amorphous form of levocetirizine dihydrochloride that is substantially free of its crystalline form and one or more pharmaceutically acceptable excipients. The pharmaceutical compositions of this aspect of the invention may be formulated, for example, as solid dosage forms for oral administration.

In accordance with yet another aspect, the invention provides a composition containing a solid form of levocetirizine dihydrochloride, which is at least 80% amorphous.

In accordance with yet another aspect, the invention provides a process for preparation of an amorphous form of levocetirizine dihydrochloride. The process is believed to be simple, eco-friendly and cost-effective. In one embodiment of this aspect of the invention, the process involves dissolution of levocetirizine in an aqueous mixture of water miscible or immiscible solvent using hydrochloric acid and further isolation by adding water immiscible aromatic or aliphatic hydrocarbon solvents. Pharmaceutical compositions that include a prophylactically or therapeutically effective amount of the amorphous form of levocetirizine dihydrochloride produced by the process described, and one or more pharmaceutically acceptable excipients are also provided.

### BRIEF DESCRIPTION OF THE ACCOMPANYING DRAWINGS

FIG. 1 is a diagram showing an X-ray powder diffraction pattern of amorphous form of levocetirizine dihydrochloride.

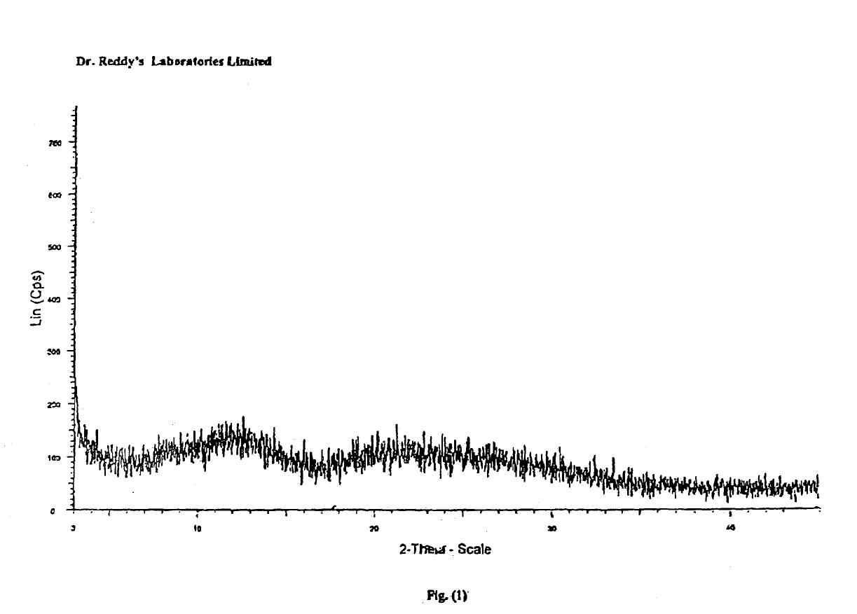

FIG. 2 shows the X-ray powder diffraction of a crystalline form of levocetirizine dihydrochloride.

FIG. 3 shows the X-ray powder diffraction of a crystalline form of dextrocetirizine dihydrochloride.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

For purposes of the present invention, the following terms are defined below.

“Pharmaceutically acceptable” means that which is useful in preparing a pharmaceutical composition that is generally non-toxic and is not biologically undesirable and includes that which is acceptable for veterinary use and/or human pharmaceutical use.

The term “composition” includes but is not limited to a solution, a suspension, a gel, an ointment, an emulsion and/or mixtures thereof. The term composition is intended to encompass a product comprising the specified ingredients in the specified amounts, as well as any product, which results, directly or indirectly, from combination of the specified ingredients in the specified amounts. A “composition” may contain a single compound or a mixture of compounds. A “compound” is a chemical substance that includes molecules of the same chemical structure.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the amorphous form of levocetirizine, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

“Therapeutically effective amount” means the amount of a compound that, when administered for treating or preventing a disease, is sufficient to effect such treatment or prevention for the disease. The “therapeutically effective amount” will vary depending on the compound, the disease and its severity and the age, weight, etc., of the patient to be treated.

When referring to a chemical reaction, the terms “treating”, “contacting” and “reacting” are used interchangeably herein and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

Cetirizine dihydrochloride is a compound of the formula:

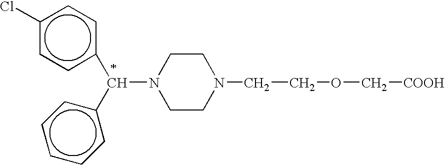

The R enantiomer is referred to as levocetirizine and the S enantiomer is referred to as dextrocetirizine. As used herein, “cetirizine” is a generic term that denotes the racemic mixture of R and S enantiomers (with respect to the asymmetric center marked with the asterisk) as well as each of the enantiomers separately. Thus, the term “substantially free of crystalline forms of cetirizine dihydrochloride,” as used herein, means that the crystalline form of cetirizine dihydrochloride cannot be detected by methods known to those skilled in the art.

The process for the preparation of levocetirizine and its salts including dihydrochloride is known. For example, GB 2 225 321 A discloses a process for preparation of levocetirizine and its dihydrochloride, which includes treating cetirizine with an acid or a base in an aqueous, alcoholic or aqueous-alcoholic medium, which is then subjected to hydrolysis and converted into levocetirizine or its dihydrochloride. The portions of the '321 patent and its U.S. counterparts, if any, which show the preparation process is/are incorporated herein by reference. An article in Tetrahedron Letters 37(28), 4837-4840 (1996), which is incorporated herein by reference, discloses the enantioselective synthesis of levocetirizine dihydrochloride and its further purification via ion exchange chromatography.

The present invention provides the amorphous form of levocetirizine dihydrochloride and the process for preparing the amorphous form of levocetirizine dihydrochloride. The inventors concluded that amorphous, free-flowing form of levocetirizine dihydrochloride is in general preferred for pharmaceutical applications because, among other reasons, it can be easily handled in pharmaceutical processing. Advantages to using the amorphous form of levocetirizine also include enhanced solubility.

FIG. (1) shows an X-ray powder diffractogram of the amorphous form of levocetirizine dihydrochloride obtained by the inventors. The X-ray powder diffraction pattern of the amorphous form of levocetirizine dihydrochloride was measured on a Bruker Axs, D8 Advance Powder X-ray Diffractometer with Cu K alpha-1Radiation source. The invention also provides a composition containing levocetirizine dihydrochloride which is at least 80% amorphous, by total weight of cetirizine dihydrochloride in the composition. The remainder of cetirizine dihydrochloride in the composition, i.e., 20% or less of the total weight of cetirizine dihydrochloride may be, for example, the crystalline form of levocetirizine dihydrochloride. In a more preferred embodiment, the composition contains at least 90% of the amorphous form with respect to total weight of levocetirizine dihydrochloride in the composition. Yet more preferably, the composition contains at least 95% of the amorphous form with respect to total weight of levocetirizine dihydrochloride in the composition. In the most preferred embodiment, the composition is substantially free of the crystalline forms of cetirizine dihydrochloride. In one preferred variant, the composition includes at least a small amount of crystalline cetirizine dihydrochloride, preferably, crystalline levocetirizine dihydrochloride. In a non-limiting example, the composition includes at least 80% of amorphous levocetirizine dihydrochloride and at least 1% crystalline levocetirizine dihydrochloride. In another non-limiting example, the composition includes at least 80% of amorphous levocetirizine dihydrochloride and at least 5% crystalline levocetirizine dihydrochloride. All compositions, in 0.1% increments, which include at least 80% of amorphous levocetirizine dihydrochloride and at least 1% crystalline levocetirizine dihydrochloride are contemplated. All percentages are based upon the total amount of cetirizine dihydrochloride in the composition.

X-ray diffraction provides a convenient and practical means for quantitative determination of the relative amounts of crystalline and amorphous forms. The X-ray powder diffraction method is capable of providing both qualitative and quantitative information about compounds present in a solid sample. X-ray diffraction is adaptable to quantitative applications because the intensities of the diffraction peaks of a given compound in a mixture are proportional to the faction of the material in the mixture.

The identification of a form of a compound from its powder diffraction pattern is based upon the position of the lines in terms of theta and their relative intensities. The diffraction angle 2 theta is determined by the spacing between a particular set of planes. Using the Bragg equation, the distance d is readily calculated from the known wavelength of the source and the measured angle.

Identification of the crystalline form is empirical. By measuring the intensity of the diffraction lines and comparing them with standards, it is possible to make a quantitative analysis of crystalline mixtures. Qualitative information can be converted to quantitative data by measuring the peak heights. Two methods that are used to analyze X-ray diffraction quantitatively are the Internal Standard Method and the External Standard Method. The Internal Standard Method is the preferred procedure for analyzing powdered systems. This method measures a known quantity of a reference powder which is added to an unknown powder. The mass absorption coefficient of the mixture need not be known in advance. Any number of constituents in the mixture may be quantified independently, including the amorphous (non-crystalline) components. The External Standard Method is used to analyze solid systems when the mass absorption co-efficient is known. It allows the quantification of one or more components in a system, which may contain an amorphous fraction.

The amount of crystalline form of levocetirizine dihydrochloride may be characterized by X-ray diffraction. The X-ray diffraction pattern for the crystalline form exhibits a diffraction pattern with a unique set of diffraction peaks that can be expressed in 2 theta angles, d-spacing values and relative peak intensities. 2 Theta diffraction angles and corresponding d-spacing values account for positions of various peaks in the X-ray powder diffraction pattern. D-spacing values are calculated with observed 2 theta angles and copper K(1) wavelength using the Bragg equation. Slight variations in observed 2 theta angles or d-spacing values are expected based on the specific diffractometer employed the analyst and the sample preparation technique. More variation is expected for the relative peak intensities. Identification of the crystal form of a compound should be based primarily on observed 2 theta angles with lesser importance attributed to relative peak intensities.

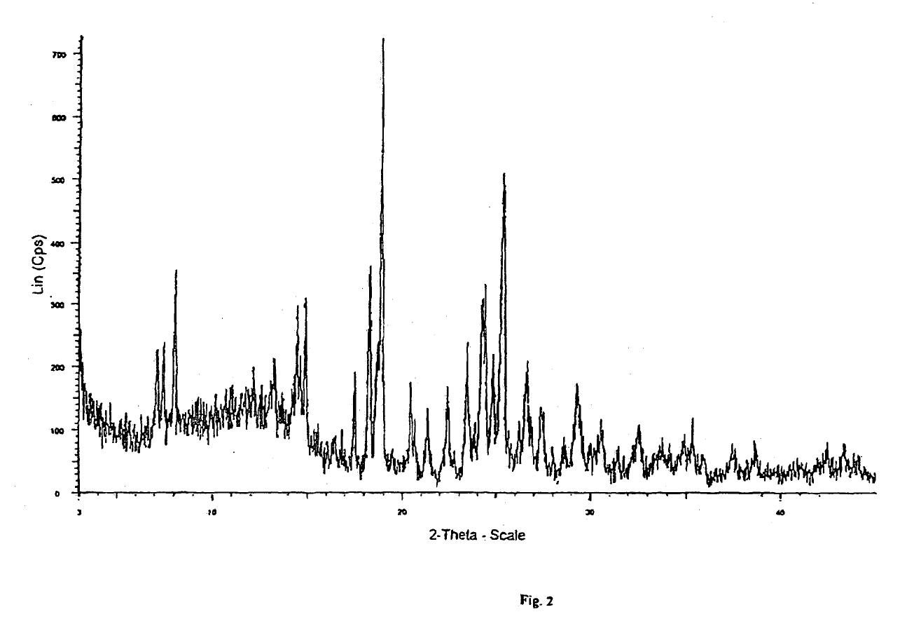

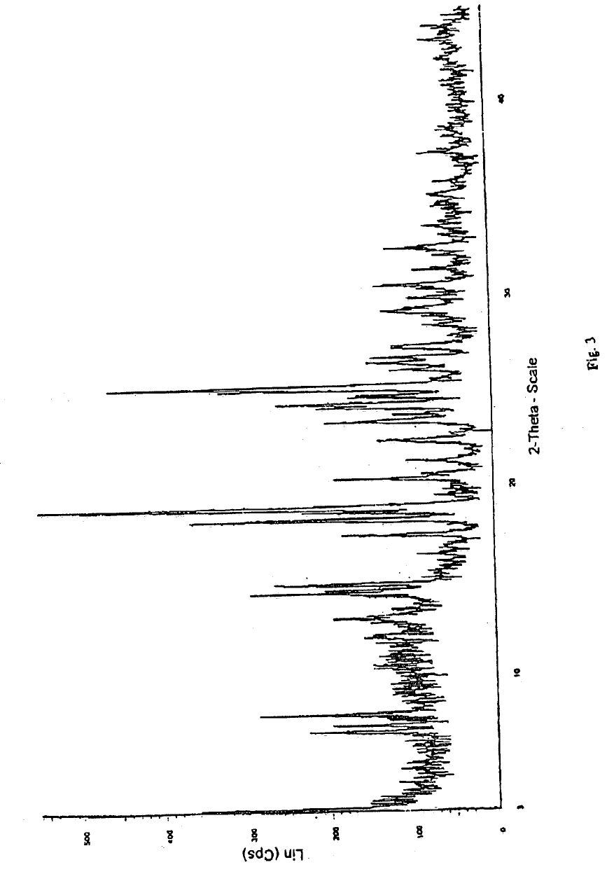

The amorphous form of levocetirizine dihydrochloride of the present invention has an X-ray powder diffractogram pattern substantially as depicted in FIG. (1). The X-ray powder diffraction pattern shows no peaks and gave a plain halo, thus demonstrating the amorphous nature of the product. For reference purposes, X-ray diffraction patterns (obtained by the inventors) of crystalline levocetirizine dihydrochloride (FIG. 2), and crystalline dextrocetirizine dihydrochloride (FIG. 3) are provided. All diffractograms-were obtained on a Bruker Axs, D8 Advance Powder X-ray Diffractometer with Cu K alpha-1 Radiation source. Table 1 below shows 2 theta and intensity values, as measured by the inventors, for the crystalline forms of cetirizine dihydrochloride and its individual enantiomers:

TABLE 1

| | | | | Cetirizine | |
|---|---|---|---|---|---|
| Dextrocetirizine | | Levocetirizine | | dihydrochloride | |
| dihydrochloride | | dihydrochloride | | (racemate) | |
| | Intensity | | Intensity | | Intensity |
| 2 theta (°) | (%) | 2 theta (°) | (%) | 2-Theta (°) | (%) |
| 18.815 | 100 | 18.855 | 100 | 18.637 | 100.0 |
| 25.247 | 73.2 | 25.311 | 79.2 | 18.244 | 81.1 |
| 18.170 | 59.5 | 18.244 | 48.9 | 25.115 | 78.8 |
| 14.805 | 35.6 | 24.211 | 41.0 | 14.423 | 47.9 |
| 24.325 | 34.6 | 24.361 | 40.5 | 17.328 | 35.9 |
| 18.591 | 29.9 | 8.018 | 37.2 | 8.007 | 28.0 |
| 14.347 | 29.0 | 14.87 | 34.2 | 20.388 | 27.8 |
| 24.158 | 28.2 | 18.648 | 30.8 | 24.143 | 25.8 |
| 7.955 | 27.1 | 23.415 | 27.5 | 7.099 | 25.4 |
| 23.354 | 27.0 | 14.408 | 26.1 | 14.731 | 22.5 |
| 17.394 | 23.4 | 26.602 | 24.7 | 23.432 | 20.7 |
| 7.053 | 23.2 | 22.388 | 21.6 | 12.966 | 20.9 |
| 20.327 | 21.7 | 17.475 | 20.6 | 22.949 | 17.8 |
| 22.330 | 19.5 | 7.096 | 19.7 | 26.109 | 16.5 |
| 24.727 | 19.0 | 24.812 | 19.5 | 29.204 | 11.3 |
| 27.347 | 17.7 | 29.282 | 19.1 | 26.706 | 10.7 |
| 30.571 | 16.8 | 7.424 | 18.8 | 8.756 | 9.9 |
| 26.514 | 16.5 | 20.42 | 18.7 | 19.965 | 9.0 |
| 26.799 | 16.3 | 27.385 | 16.1 | 15.923 | 8.8 |

The percent composition of crystalline levocetirizine can be determined in an unknown composition. The X-ray powder diffraction patterns of an unknown composition can be compared to a known standard containing pure crystalline levocetirizine to identify the percent ratio of the crystalline form of levocetirizine dihydrochloride. This is done by comparing the relative intensities of the peaks from the diffraction pattern of the unknown composition with a calibration curve derived from the X-ray diffraction pattern of a pure crystalline sample of levocetirizine. The curve can be calibrated based on the X-ray powder diffraction pattern for the strongest peak from a pure sample of crystalline levocetirizine. The peak intensities are reported as intensities relative to the peak intensity of the strongest peak (“the 100% peak”). The 100% peak for cetirizine dihydrochloride at 2-theta ˜18.64, for levocetirizine dihydrochloride at ˜18.85, for dextrocetirizine dihydrochloride at ˜18.81 (TABLE 1).

The calibration curve may be created in a manner known to those of skill in the art. For example, five or more artificial mixtures of amorphous and crystalline forms of crystalline levocetirizine dihydrochloride, at different amounts, may be prepared. In a non-limiting example, such mixtures may contain, 2%, 5%, 7%, 8%, and 10% of crystalline levocetirizine dihydrochloride, with the remainder being the amorphous form of the salt. Then, X-ray diffraction patterns are obtained for each artificial mixture using standard X-ray diffraction techniques. Slight variations in peak positions, if any, may be accounted for by adjusting the location of the peak to be measured. The intensities of the 100% peak(s) for each of the artificial mixtures are then plotted against the known weight percentages of the crystalline form of the salt. The resulting plot is a calibration curve that allows determination of the amount of crystalline cetirizine dihydrochloride in an unknown sample. For the unknown mixture of crystalline and amorphous levocetirizine dihydrochloride, the intensities of the 100% peak(s) in the mixture, relative to an intensity of this peak in a calibration mixture, may be used to determine the percentage of the crystalline form in the composition, with the remainder determined to be the amorphous material.

The invention also provides a process for preparation of amorphous levocetirizine dihydrochloride. The starting material for preparation of amorphous cetirizine dihydrochloride may be cetirizine free base or salt other than dihydrochloride. In this case, the starting material is suspended or dissolved in a solvent carrier and a suitable amount of hydrochloric acid is added to convert the starting material to the dihydrochloride salt. If the starting material is dihydrochloride salt of levocetirizine (e.g., crystalline or oil form), addition of hydrochloric acid may be unnecessary. The solvent carrier may be a mixture of water with an organic solvent. If the starting material is cetirizine free base, it may be suspended in the water-based solvent carrier and dissolves as the dihydrochloride salt is formed upon addition of the hydrochloric acid. Then, the solvent is removed, for example, by evaporation under vacuum or otherwise to obtain a residue of dihydrochloric salt, which is then triturated with hydrocarbon solvent.

In one specific embodiment, the amorphous form of levocetirizine dihydrochloride may be prepared, for example, by

(i) providing levocetirizine free base or salt thereof in a solvent carrier,

(ii) treating the levocetirizine in said carrier with hydrochloric acid;

(iii) removing the solvent carrier to obtain a residue;

(iv) adding water immiscible aromatic or aliphatic or alicyclic hydrocarbon solvents such as toluene, xylene, cyclohexane or heptane, preferably cyclohexane to said residue thereby said amorphous form of levocetirizine dihydrochloride separates as a solid mass;

(v) filtering the compound;

(vi) drying the compound to isolate the desired amorphous form of levocetirizine dihydrochloride.

Examples of solvent carriers include, but are not limited to, water; a ketone solvent, such as acetone, methyl ethyl ketone, 2-pentanone or a mixture thereof; a mixture of water and water-miscible solvents like C₁-C₅ straight or branched chain alcoholic solvents (e.g., methanol, ethanol, n-propanol, isopropanol, 2-butanol, n-butanol, n-pentanol or 2-pentanol); a nitrile solvent, such as acetonitrile or propionitrile; and water immiscible aromatic or aliphatic or alicyclic hydrocarbon solvent, such as toluene, cyclohexane or heptane. Acetone, isopropanol, acetonitrile, and toluene are preferred.

The amorphous form of levocetirizine dihydrochloride described herein is thermally stable and may be used as an active ingredient in pharmaceutical formulations. The pharmaceutical compositions of the invention contain the amorphous form of levocetirizine dihydrochloride as the active ingredient, and one or more pharmaceutically acceptable excipients. Suitable pharmaceutically acceptable excipients include starches, sugars, celluloses, such as microcrystalline cellulose, hydroxypropyl cellulose, and hydroxypropylmethyl cellulose, diluents, granulating agents, lubricants, binders, disintegrating agents, and the like.

The amorphous form of the composition comprising levocetirizine dihydrochloride has a moisture content which varies from 0.3 to 12.0% by KF method. Typically, the moisture content of the substance is around 1.5 to 7.5% by KF method. The moisture content of present inventive substance was measured on Mettler DL-35 instrument using Karl-Fischer reagent.

Generally, the pharmaceutical compositions of the present invention are prepared by uniformly admixing the active ingredient with liquid or solid carriers and then shaping the product into the desired form. The pharmaceutical compositions may be in the form of suspensions, solutions, elixirs, aerosols, or solid dosage forms. Because of their ease of administration, tablets and capsules represent the most advantageous oral dosage unit form, in which case solid pharmaceutical carriers are employed. Examples of formulations suitable for the amorphous form of levocetirizine dihydrochloride of the invention are disclosed in U.S. Pat. Nos. 6,245,353 and 5,698,558, the disclosures of which are incorporated herein by reference in their entirety.

The more preferred oral solid preparation is a tablet. A tablet may be prepared by direct compression, wet granulation, or molding, of the amorphous form of levocetirizine dihydrochloride with a carrier and other excipients in a manner known to those skilled in the art. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made on a suitable machine, a mixture of the powdered compound moistened with an inert liquid diluent, are suitable in the case of oral solid dosage forms (e.g., powders, capsules, and tablets). If desired, tablets may be coated by standard techniques. The amorphous form of levocetirizine dihydrochloride described herein may be formulated into typical disintegrating tablet, or into a controlled or extended release dosage forms. Examples of suitable controlled release formulation vehicles are disclosed in U.S. Pat. Nos. 3,845,770; 3,916,899; 3,536,809; 3,598,123; and 4,008,719, the disclosures of which are hereby incorporated by reference in their entirety. U.S. Pat. No. 5,698,558, incorporated by reference in its entirety, discloses a method of utilizing levocetirizine, or a pharmaceutically acceptable salt thereof, substantially free of its (+) stereoisomer.

Preferably, each tablet contains from about 2 mg to about 10 mg of the amorphous form of levocetirizine dihydrochloride, and each cachet or capsule contains from about 2 mg to about 10 mg of the amorphous form of levocetirizine dihydrochloride. Most preferably, the tablet contains about 2 mg, about 5 mg or about 10 mg of the amorphous form of levocetirizine dihydrochloride for oral administration.

The prophylactic or therapeutic dose of the amorphous form of levocetirizine dihydrochloride will vary with the severity of the condition to be treated and the route of administration. The dose and perhaps the dose frequency will also vary according to the age, body weight and response of the individual patient. In general, the total daily dose range for the amorphous form of levocetirizine dihydrochloride is from about 1.0 mg to about 25 mg. Preferably, a daily dose range should be about 2.0 mg to about 20 mg in single or divided doses; most preferably, the dose range is from about 5 mg to about 10 mg per day. It is known that children and elderly patients, as well as those with impaired renal or hepatic function, should receive low doses, at least initially.

The term “prophylactically or therapeutically effective amount” refers to the above-described dosage amounts and dose frequency schedules. Any suitable route of administration may be employed. For example, oral, rectal, parenteral (subcutaneous, intramuscular, intravenous), and transdermal, and like forms of administration may be suitable. Oral route of administration is preferred.

Hence, the present invention is directed to provide the amorphous form of levocetirizine dihydrochloride. The process for the preparation of present invention is simple, eco-friendly and commercially viable.

### EXAMPLES

The invention is further defined by reference to the following examples describing in detail the preparation of the compound and the compositions of the present invention, as well as their utility. It will be apparent to those skilled in the art, that many modifications, both to materials, and methods, may be practiced without departing from the purpose and interest of this invention.

### REFERENCE EXAMPLE

Preparation of Levocetirizine:

(+)-[2-[4-[(4-Chlorophenyl)-phenyl methyl]-1-piperazinyl]ethanol (105 grams) was dissolved in dimethyl formamide (357 ml) and cooled to a temperature of 0-5° C. Potassium hydroxide (53.3 grams) was added to the reaction mixture and maintained for 90 minutes. Sodium monochloroacetate (55.5 grams) was then added and further maintained at a temperature of 0-5° C. for 90 minutes. The temperature of the reaction mixture was raised to 30-35° C. and maintained until the reaction was substantially complete. Water (1155 ml) was added to the reaction mixture and the pH of the reaction mixture was adjusted to 9.5-9.8 with hydrochloric acid. The reaction mixture was then washed with ethyl acetate (760 ml) and the layers were separated. The pH of the aqueous layer was adjusted to 4-4.5 with Hydrochloric acid and extracted with dichloromethane (875 ml). The extracted organic layer was first washed with 10% Sodium chloride solution, and then washed with water. The solvent was distilled off from the reaction solution to afford levocetirizine (Weight: 123.0 grams).

### Example 1

Levocetirizine (5 grams) was dissolved in a mixture of water (20 ml) and acetone (50 ml) at room temperature. Hydrochloric acid (5 ml) was added to the reaction mixture and the solution was stirred for a period of 10 to 30 minutes. Then, the reaction solution was then filtered and the solvent was completely distilled off to dryness at a temperature below 80° C. Cyclohexane (50 ml) was added to the residual mass and stirred for 30 minutes at a temperature of 30-35° C. The product was then filtered and washed with cyclohexane (25 ml) and subsequently dried at a temperature of 60-110° C. to a constant weight to afford the amorphous form of levocetirizine dihydrochloride. Weight: 4.7 grams, M.C by KF: 1.7%.

### Example 2

Levocetirizine (10.0 grams) was taken in a mixture of toluene (100 ml) and water (50 ml) at room temperature. Concentrated hydrochloric acid solution (10 ml) was added to the reaction mixture and the solution was stirred to get the clear solution. Then, the reaction solution was filtered and the solvent was completely distilled off to dryness at a temperature of 70-90° C. under vacuum. Toluene (100 ml) was added to the residual mass and stirred for 30 minutes at a temperature of 30-35° C. The compound was then filtered and washed with toluene (50 ml). Subsequently, the compound was dried at a temperature of 60-65° C. to a constant weight to afford the amorphous form of levocetirizine dihydrochloride. Weight: 9.4 grams, M.C by KF: 4.0%.

### Example 3

Levocetirizine (10.0 grams) was taken in a mixture of acetonitrile (100 ml) and water (50 ml) at room temperature. Concentrated hydrochloric acid solution (6.0 ml) was added to the reaction mixture and the solution was stirred to get the clear solution. The reaction solution was then filtered. The solvent was completely distilled off to dryness at a temperature of 70-80° C. under vacuum to afford the amorphous form of levocetirizine dihydrochloride. Weight: 12.0 grams; M.C by KF: 2.3%.

### Example 4

Levocetirizine dihydrochloride (1.0 grams) was dissolved in a mixture of acetone (40 ml) and water (100 ml). The reaction mixture was stirred at a temperature of 25-35° C. to get a clear solution. The reaction solution was then filtered and the solvent was completely distilled off from the reaction solution to dryness at a temperature of 50-75° C. under reduced pressure to result the amorphous form of levocetirizine dihydrochloride. The amorphous form of levocetirizine dihydrochloride was further dried at a temperature of 70-75° C. to a constant weight to afford the amorphous form of levocetirizine dihydrochloride. Weight: 9.4 grams; M.C by KF: 5.8%.

### Example 5

Levocetirizine dihydrochloride (10.0 grams) was dissolved in water (30 ml) at a temperature of 25-35° C. Toluene (100 ml) was added to the reaction solution and the solvent was completely distilled off to dryness from the reaction solution at a temperature of 60-80° C. Cyclohexane (200 ml) was then added to the residual mass, which was then stirred for 45-60 minutes at a temperature of 25-35° C. to crystallize the solid mass. The separated solid was filtered and washed with cyclohexane (50 ml). The solid was then dried at a temperature of 60-70° C. to a constant weight to afford the amorphous form of levocetirizine dihydrochloride. Weight: 9.6 grams; M.C by KF 3.5%.

### Example 6

Levocetirizine dihydrochloride (15.0 grams) was dissolved in water (15 ml) at a temperature of 25-35° C. Isopropanol (150 ml) was then added to the reaction solution and the solvent was completely distilled off to dryness from the reaction solution at a temperature of 70-80° C. Then di isopropyl ether (300 ml) was added to the residual mass and stirred for 45-60 minutes at a temperature of 25-35° C. to crystallize the solid mass. The separated solid was filtered and then washed with di isopropyl ether (75 ml). The solid was then dried at a temperature of 60-75° C. to a constant weight to afford the amorphous form of levocetirizine dihydrochloride. Weight: 14.8 grams; M.C by KF 4.6%.

### Example 7

Levocetirizine (10.0 grams) was dissolved in ethyl acetate (100 ml) at a temperature of 25-35° C. and stirred for 10-15 min. Isopropanolic hydrochloric acid (20 ml) was added till the pH of reaction mass becomes 2.0. The reaction mass was stirred for 1-2 hours to separate the solid. The separated solid was filtered, washed with ethyl acetate (20 ml), followed by hexane (10 ml) and on subsequent drying at a temperature of 80-100° C. to a constant weight resulted the novel amorphous form of levocetirizine dihydrochloride. Weight: 10.2 grams.

### Example 8

Soluble Granules Containing Amorphous Levocetirizine Dihydrochloride

Soluble granules containing amorphous levocetirizine dihydrochloride may have the following content:

| Ingredient | Content (mg) |
|---|---|
| Amorphous levocetirizine | 10 |
| dihydrochloride | |
| Calcium carbonate | 800 |
| Citric acid | 900 |
| Avicel | 40 |
| Mannitol | 625 |
| Maltodextrin | 15 |
| Aspartame | 3 |
| Aroma | 20 |

### Example 9

Dispersible Tablet Containing Levocetirizine Dihydrochloride

Dispersible tablet containing amorphous levocetirizine dihydrochloride may have the following content:

| Ingredient | Content (mg) |
|---|---|
| Amorphous levocetirizine | 10 |
| dihydrochloride | |
| Calcium carbonate | 500 |
| Polyvinylpyrrolidone | 17 |
| Avicel | 15 |
| Mannitol | 400 |
| Maltodextrin | 15 |
| Aspartame | 3 |
| Aroma | 20 |

### Example 10

Preparation of Crystalline Standard of levocetirizine dihydrochloride

Levocetirizine (10.0 grams) was dissolved in ethyl acetate (100 ml) at a temperature of 25-35° C. and stirred for 10-15 min. Isopropanolic hydrochloric acid (20 ml) was added till the pH of reaction mass becomes 2.0. The reaction mass was stirred for 1-2 hours to separate the solid. The separated solid was filtered, washed with ethyl acetate (20 ml), followed by hexane (10 ml) and on subsequent drying at a temperature of 80-100° C. to a constant weight resulted the novel crystalline Form -I of Cetirizine dihydrochloride (Weight: 10.2 grams).

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

We claim:

1. An amorphous form of levocetirizine dihydrochloride.

2. An amorphous form of levocetirizine dihydrochloride, which is substantially free of crystalline forms of cetirizine dihydrochloride.

3. An amorphous form of levocetirizine dihydrochloride characterized by an X-ray powder diffraction pattern substantially in accordance with FIG. (1).

4. A pharmaceutical composition comprising a prophylactically or therapeutically effective amount of an amorphous form of levocetirizine dihydrochloride and one or more pharmaceutically acceptable excipients.

5. The pharmaceutical composition of claim 4, which is substantially free of crystalline forms of cetirizine dihydrochloride.

6. A composition comprising levocetirizine dihydrochloride as a solid, wherein at least 80% by weight of said levocetirizine dihydrochloride is in an amorphous form.

7. The composition of claim 5, wherein at least 90% of said solid levocetirizine dihydrochloride is in an amorphous form.

8. The composition of claim 6, wherein at least 95% of said solid levocetirizine dihydrochloride is in an amorphous form.

9. The composition of claim 7, wherein at least 99% of said solid levocetirizine dihydrochloride is in an amorphous form.

10. The composition of claim 8, which is substantially free of crystalline forms of cetirizine dihydrochloride.

11. The composition of claim 6, wherein at least 1% of said solid levocetirizine dihydrochloride is in a crystalline form.

12. The composition of claim 11, wherein at least 5% of said solid levocetirizine dihydrochloride is in a crystalline form.

13. The composition of claim 6, which is a pharmaceutical composition.

14. The composition of claim 13, further comprising one or more pharmaceutically acceptable excipients.

15. The composition of claim 14, wherein said pharmaceutical composition is a solid dosage form for oral administration.

16. The composition of claim 15, wherein said solid dosage form is a tablet.

17. The composition of claim 6 having a moisture content ranging from about 0.3% to about 12% by KF method.

18. The composition of claim 6 having a moisture content ranging from about 1.5% to about 7.5% by KF method.

19. A process for the preparation of an amorphous form of (−)-[2-[4-[(4-Chlorophenyl)-phenyl methyl]-1-piperazinyl]ethoxy] acetic acid dihydrochloride (levocetirizine dihydrochloride), which comprises
a) providing levocetirizine free base or salt thereof in a solvent carrier;
b) treating said levocetirizine in said carrier with hydrochloric acid to form a dihydrochloride salt of cetirizine in solution;
c) removing said solvent carrier to obtain a residue;
d) adding a liquid hydrocarbon compound to said residue thereby said amorphous form of levocetirizine dihydrochloride separates as a solid mass.

20. The process of claim 19, further comprising isolating said solid mass.

21. The process of claim 20, further comprising removing any unbound solvent from said isolated solid mass to obtain a substantially dry form of said amorphous form of levocetirizine dihydrochloride.

22. The process of claim 21, wherein said step of removing said unbound solvent comprises drying said solid mass at a temperature of from about 60 to about 110 degrees Celsius.

23. The process of claim 22, further comprising removing any unbound solvent from said isolated solid mass to obtain a substantially dry form of said amorphous form of levocetirizine dihydrochloride.

24. The process of claim 19, wherein said liquid hydrocarbon compound is selected from a group consisting of toluene, xylene, cyclohexane, or heptane.

25. The process of claim 19, wherein said solvent carrier is selected from a group consisting of a ketone solvent, an aqueous mixture of water miscible solvents, a nitrile solvent, or a hydrocarbon solvent.

26. The process of claim 25, wherein said ketone solvent is selected from a group consisting of acetone, methyl ethyl ketone or 2-pentanone or mixture thereof.

27. The process of claim 25, wherein said aqueous mixture of water miscible solvents is a C₁-C₅ straight or branched chain alcoholic solvent.

28. The process of claim 27, wherein the branched chain alcoholic solvent is selected from the group consisting of methanol, ethanol, n-propanol, isopropanol, 2-butanol, n-butanol, n-pentanol or 2-pentanol.

29. The process of claim 25, wherein said nitrile solvent is acetonitrile or propionitrile.

30. The amorphous form of levocetirizine dihydrochloride produced in accordance with a process of claim 19.

31. The amorphous form of levocetirizine dihydrochloride produced in accordance with a process of claim 22.

32. The amorphous form of levocetirizine dihydrochloride produced in accordance with a process of claim 25.

33. A pharmaceutical composition comprising i) a prophylactically or therapeutically effective amount of levocetirizine dihydrochloride in a solid form produced by the process of claim 19, and ii) one or more pharmaceutically acceptable excipients.

34. The composition of claim 33, wherein said pharmaceutical composition is a solid dosage form for oral administration.

35. The composition of claim 34, wherein said solid dosage form is a tablet.

36. The composition of claim 33, having a moisture content ranging from about 0.3% to about 12% by KF method.

37. The composition of claim 33, having a moisture content ranging from about 1.5% to about 7.5% by KF method.
